(19) United States
(12) Patent Application Publication
(10) Pub. No.: US 2025/0099354 A1
(43) Pub. Date: Mar. 27, 2025

(54) COSMETIC COMPOSITION COMPRISING AT LEAST ONE FATTY AMINE, AT LEAST 6% BY WEIGHT OF AT LEAST ONE SOLID FATTY SUBSTANCE AND AT LEAST ONE LIQUID FATTY SUBSTANCE

(71) Applicant: L'Oreal, Paris (FR)

(72) Inventors: Cécile Toulouzan, Saint Ouen (FR); Virginie Le Chaux, Saint Ouen (FR); Estelle Millet, Saint Ouen (FR)

(73) Assignee: L'Oreal, Paris (FR)

(21) Appl. No.: 18/580,952

(22) PCT Filed: Jul. 19, 2022
(86) PCT No.: PCT/EP2022/070132

§ 371 Date: Jan. 19, 2024

(30) Foreign Application Priority Data
Jul. 22, 2021 (FR) 2107907

(51) Int. Cl. A61K 8/42; A61Q 5/00; A61Q 5/12

(52) U.S. Cl. CPC A61K 8/42; A61Q 5/002; A61Q 5/12; A61K 2800/592

(57) ABSTRACT

The present invention relates to a cosmetic composition comprising at least one fatty amine containing at least one C₈-C₃₀ hydrocarbon-based chain, at least 6% by weight of at least one fatty substance with a melting point above 25° C. including a particular solid ester of carboxylic acid and monoalcohol, and at least one fatty substance with a melting point below or equal to 25° C. The invention also relates to a process for the cosmetic treatment of keratin fibres, in which such a composition is applied to said fibres, and also to the use of this composition for caring for keratin fibres, in particular hair. The invention also relates to the use of the composition according to the invention for conditioning keratin fibres, in particular hair.

The present invention relates to a cosmetic composition comprising at least one fatty amine containing at least one C₈-C₃₀ hydrocarbon-based chain, at least 6% by weight of at least one fatty substance with a melting point above 25° C., including a particular solid ester of carboxylic acid and monoalcohol, and at least one fatty substance with a melting point below or equal to 25° C.

The invention also relates to a process for the cosmetic treatment of keratin fibres, in which such a composition is applied to said fibres, and also to the use of this composition for caring for keratin fibres, in particular hair.

The invention also relates to the use of the composition according to the invention for conditioning keratin fibres, in particular hair.

Hair is generally damaged and embrittled by the action of external atmospheric agents such as light and bad weather, and also by mechanical or chemical treatments, such as brushing, combing, dyeing, bleaching, permanent-waving and/or relaxing, or even repeated washing.

Hair is thus damaged by these various factors and may over time become dry, coarse, brittle or dull, notably in fragile areas, and more particularly at the ends.

Thus, to overcome these drawbacks, it is common practice to resort to haircare treatments using compositions that condition the hair, giving it satisfactory cosmetic properties, notably in terms of smoothness, sheen, softness, suppleness, lightness, a natural feel and good disentangling properties.

These haircare compositions intended to be applied regularly to the hair may be, for example, hair conditioners, masks or sera, and may be in the form of gels, hair lotions or care creams that are more or less thick.

It is in particular known practice to use compositions in the form of oily sera, containing combinations of silicone oils or non-silicone oils.

However, such compositions still too often result in excessive greasing of keratin fibres, which results in said fibres having a greasy or even tacky feel, and a lank, weighed-down visual appearance.

Furthermore, compositions containing silicone oils, once applied to the hair, frequently result in effects that are not very pleasant to the touch, in particular the hair has a tendency to be coarse and/or to squeak (that is to say produce an unpleasant sound), in particular when the strands of hair are rubbed together while sliding the fingers along the hair from the root to the end.

In addition, hair treated with such compositions is neither easy nor quick to dry.

The present invention aims to provide compositions which do not have the drawbacks mentioned above, and which are capable of conditioning keratin fibres in a long-lasting manner, in particular by giving them good cosmetic properties.

This aim is achieved by the present invention, one subject of which is notably a cosmetic composition comprising:
(i) one or more fatty amines containing at least one C₈-C₃₀ hydrocarbon-based chain,
(ii) at least 6% by weight, relative to the total weight of the composition, of one or more fatty substances with a melting point above 25° C., different from the fatty amines (i), and
(ii) one or more fatty substances with a melting point below or equal to 25° C., different from the fatty amines (i),
the composition comprising one or more fatty substances with a melting point above 25° C. (ii) chosen from esters of a linear or branched, saturated carboxylic acid including at least 10 carbon atoms and of a linear or branched, saturated monoalcohol, including at least 10 carbon atoms.

The composition according to the invention makes it possible to impart good cosmetic properties to the keratin fibres, in particular to the hair, notably as regards disentangling, smooth feel and smooth appearance, softness, sheen, manageability and frizz control.

Moreover, the composition according to the invention has good working qualities. It is easy to apply and to distribute over the keratin fibres. It has a pleasant texture. It is removed very well, by simple rinsing of the keratin fibres, without leaving a greasy feel or appearance of these fibres. The fibres are light, not laden or tacky.

The composition makes it possible to obtain a composition, notably a natural hair care composition (based on ingredients of natural origin) that makes it possible to obtain the same working and conditioning qualities, such as disentangling and smooth feel and smooth appearance, as a non-natural care product comprising, for example, silicones.

Another subject of the invention is a process for the cosmetic treatment of human keratin fibres such as the hair, comprising the application to said keratin fibres of a composition as defined above.

Another subject of the present invention relates to the use of the composition as defined above for conditioning keratin fibres, in particular human keratin fibres such as the hair.

Other subjects, characteristics, aspects and advantages of the invention will become even more clearly apparent on reading the description and the examples.

In the present description, and unless otherwise indicated:
the expression “at least one” is equivalent to the expression “one or more” and can be replaced therewith;
the expression “between” is equivalent to the expression “ranging from” and can be replaced therewith, and implies that the limits are included;
according to the present application, the term “keratin fibres” denotes human keratin fibres, and more particularly the hair.

For the purposes of the present invention, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name MDSC 2920 by the company TA Instruments. In the present application, all the melting points are determined at atmospheric pressure (1.013×10⁵ Pa).

The term “fatty substance” means an organic compound that is insoluble in water at 25° C. and at atmospheric pressure (1.013×10⁵ Pa) (solubility of less than 5% by weight, preferably less than 1% by weight and even more preferentially less than 0.1% by weight). The fatty substances have in their structure at least one hydrocarbon-based chain including at least 6 carbon atoms and/or a sequence of at least two siloxane groups. In addition, the fatty substances are generally soluble in organic solvents under the same temperature and pressure conditions, for instance chloroform, dichloromethane, carbon tetrachloride, ethanol, benzene, toluene, tetrahydrofuran (THF), liquid petroleum jelly or decamethylcyclopentasiloxane.

Preferably, the composition according to the invention is free of silicones. For the purposes of the present invention, the term “free of silicones” refers to a composition which contains less than 0.05% by weight of silicones, relative to the total weight of the composition, and preferably does not contain silicones (0% by weight, relative to the total weight of the composition).

Fatty Amines Containing at Least One C₈-C₃₀ Hydrocarbon-Based Chain (i)

The composition according to the invention comprises one or more fatty amines containing at least one C₈-C₃₀ hydrocarbon-based chain (i), that is to say a hydrocarbon-based chain comprising from 8 to 30 carbon atoms.

Preferably, the composition according to the invention comprises one or more fatty amines (i) chosen from fatty amidoamines containing at least one C₈-C₃₀ hydrocarbon-based chain, that is to say a hydrocarbon-based chain comprising from 8 to 30 carbon atoms.

Preferably, the fatty amines are chosen from (C₁₀-C₃₀) alkylamido (C₁-C₅) alkyl (di)(C₁-C₆) alkylamines, salts thereof and solvates thereof.

In the term (C₁₀-C₃₀) alkylamido (C₁-C₈) alkyl (di)(C₁-C₆) alkylamine it is understood that the alkyl group may be linear or branched and the term amido should be understood as the group —C(O)—N(R)— or —N(R)—C(O)— where R represents a hydrogen atom or a linear or branched (C₁-C₆) alkyl. In particular, the (C₁₀-C₃₀) alkylamido (C₁-C₈) alkyl (di)(C₁-C₆) alkylamine of the invention corresponds to the formula (A):

R¹—C(O)—N(H)—R²—NR³—R⁴ (A)

in which:
R¹ is a linear C₁₀-C₃₀ alkyl group,
R² is a linear C₁ to C₈ alkylene group, and
R³ and R⁴, which may be identical or different, are linear alkyl groups having a C₁ to C₆ alkyl chain.

Preferably, in formula (A):
R¹ is a linear alkyl group having a C₁₄-C₂₆ carbon-based chain, better still R¹ is a linear alkyl group having a C₁₆-C₂₄ carbon-based chain, even better still R¹ is a linear alkyl group having a C₁₈-C₂₂ carbon-based chain.

Preferably, in formula (A):
R² is a linear C₁ to C₆ alkylene group, better still R² is a linear C₂ to C₄ alkylene group, even better still R² is a —(CH₂)₃— group.

Preferably, in formula (A):
R³ and R⁴, which may be identical or different, are linear alkyl groups having a C₁ to C₄ carbon chain; better still R³ and R⁴ are identical and represent methyl or ethyl; even better still R³ and R⁴ are —CH₃ groups.

The cosmetic salts and the solvates such as the hydrates of the tertiary substituted amidoamines are also used according to the present invention.

The fatty amines according to the invention may be present in quaternized form, in particular in the form of halide salts such as chloride or bromide, or alkylsulfate salt such as methosulfate.

Preferably, the fatty amines according to the invention, in particular the fatty amidoamines according to the invention, are not quaternized.

The compounds of formula (A) are preferably chosen from:
the brassicamidopropyl dimethylamine of formula (A1) below:

R—C(O)—N(H)—(CH₂)₃—N(CH₃)₂ (A1)

in which R—C(O) is a fatty acid derived from Brassica campestris seed oil (rapeseed oil), with a majority of behenyl (C₂₂) groups.

Among these compounds of formula (A1), mention may be made of ProCondition™ 22 from Inolex Chemical Company.
the stearylamidopropyl dimethylamine of formula (A2) below:

CH₃—(CH₂)₁₆—C(O)—N(H)—(CH₂)₃—N(CH₃)₂ (A2)

Among these compounds of formula (A2), mention may notably be made of TEGO Amid S 18 from Evonik and Genamin SPA from Clariant.
the behenamidopropyl dimethylamine of formula (A3) below:

CH₃—(CH₂)₂₀—C(O)—N(H)—(CH₂)₃—N(CH₃)₂ (A3)

Among these compounds of formula (A3), mention may notably be made of Amidet APA-22 provided by Kao, or Mackine 601 from Solvay.

Preferentially, the fatty amine(s) containing at least one C₈-C₃₀ hydrocarbon-based chain (i) are chosen from (C₁₄-C₂₆) alkylamido (C₁-C₆) alkyl (di)(C₁-C₄) alkylamines, salts thereof and solvates thereof; more preferentially from (C₁₆-C₂₄) alkylamido (C₂-C₄) alkyl (di)(C₁-C₂) alkylamines, salts thereof and solvates thereof; still from (C₁₈-C₂₂) alkylamido (C₂-C₄) alkyl (di)(C₁-30 more preferentially C₂) alkylamines, salts thereof and solvates thereof; better still from brassicamidopropyl dimethylamine, stearamidopropyl dimethylamine, behenamidopropyl dimethylamine and mixtures thereof.

Advantageously, in the composition according to the invention, the total content of the fatty amine(s) containing at least one C₈-C₃₀ hydrocarbon-based chain (i) is greater than or equal to 2% by weight, preferably ranges from 2% to 10% by weight, more preferentially ranges from 2% to 6% by weight, relative to the total weight of the composition.

Preferably, the total content of the (C₁₀-C₃₀) alkylamido (C₁-C₈) alkyl (di)(C₁-C₆) alkylamines, salts thereof and solvates thereof is greater than or equal to 2% by weight, preferably ranges from 2% to 10% by weight, more preferentially ranges from 2% to 6% by weight, relative to the total weight of the composition.

Preferably, the total content of the (C₁₀-C₃₀) alkylamido (C₁-C₈) alkyl (di)(C₁-C₆) alkylamines chosen from brassicamidopropyl dimethylamine, stearamidopropyl dimethylamine, behenamidopropyl dimethylamine, and mixtures thereof is greater than or equal to 2% by weight, preferably ranges from 2% to 10% by weight, more preferentially ranges from 2% to 6% by weight, relative to the total weight of the composition.

### Solid Fatty Substances (ii)

The cosmetic composition according to the present invention comprises at least 6% by weight, relative to the total waste the composition, of at least one fatty substance (ii), different from the fatty amines (i), with a melting point above 25° C., preferably above or equal to 28° C., more preferentially above or equal to 30° C. at atmospheric pressure (1.013×10⁵ Pa). In the present patent application, this or these fatty substances are also referred to as “solid fatty substance(s)”.

### Solid Fatty Substances (ii): Solid Esters of Carboxylic Acid and of Monoalcohol

The cosmetic composition according to the present invention comprises one or more fatty substances with a melting point above 25° C. (ii) chosen from esters of a linear or branched, saturated carboxylic acid including at least 10 carbon atoms and of a linear or branched, saturated monoalcohol, including at least 10 carbon atoms.

Advantageously, the solid esters of carboxylic acid and of monoalcohol that can be used in the present invention are neither (poly) oxyalkylenated nor (poly)glycerolated.

Preferably, the solid esters of carboxylic acid and of monoalcohol are non-silicone substances.

Preferably, these solid fatty esters are esters of a linear or branched, saturated carboxylic acid including from 10 to 30 carbon atoms and preferably from 12 to 24 carbon atoms, and of a linear or branched, saturated monoalcohol, including from 10 to 30 carbon atoms and preferably from 12 to 24 carbon atoms. The saturated carboxylic acids may optionally be hydroxylated, and are preferably monocarboxylic acids.

Mention may notably be made of octyldodecyl behenate, isocetyl behenate, myristyl stearate, cetyl stearate, stearyl stearate, cetyl myristate, myristyl myristate, stearyl myristate, myristyl palmitate, cetyl palmitate, stearyl palmitate, and mixtures thereof.

Preferably, the solid esters of a linear or branched, saturated carboxylic acid including at least 10 carbon atoms and of a linear or branched, saturated monoalcohol, including at least 10 carbon atoms are chosen from C₁₀-C₂₆ alkyl palmitates, notably myristyl palmitate, cetyl palmitate and stearyl palmitate; C₁₀-C₂₆ alkyl myristates, such as cetyl myristate, stearyl myristate and myristyl myristate; C₁₀-C₂₆ alkyl stearates, notably myristyl stearate, cetyl stearate and stearyl stearate; and mixtures thereof.

Particularly preferably, the solid esters of a linear or branched, saturated carboxylic acid including at least 10 carbon atoms and of a linear or branched, saturated monoalcohol, including at least 10 carbon atoms are chosen from myristyl stearate, cetyl stearate, stearyl stearate, cetyl myristate, myristyl myristate, stearyl myristate, myristyl palmitate, cetyl palmitate, stearyl palmitate, and mixtures thereof.

### Solid Fatty Substances (ii): Additional Solid Fatty Substances (ii)

The cosmetic composition according to the present invention may also comprise one or more additional fatty substances with a melting point above 25° C. (ii) which are different from the esters of a linear or branched, saturated carboxylic acid including at least 10 carbon atoms and of a linear or branched, saturated monoalcohol, including at least 10 carbon atoms.

Advantageously, the solid fatty substances that can be used in the present invention are neither (poly) oxyalkylenated nor (poly) glycerolated.

The solid fatty substances according to the invention preferably have a viscosity of greater than 2 Pa·s, measured at 25° C. and at a shear rate of 1 s⁻¹.

Preferably, the solid fatty substances are non-silicone substances.

The fatty substance(s) with a melting point above 25° C. (ii) are preferably chosen from solid fatty acids, solid fatty alcohols, solid esters of fatty acids and/or of fatty alcohols different from esters of a linear or branched, saturated carboxylic acid including at least 10 carbon atoms and of a linear or branched, saturated monoalcohol, including at least 10 carbon atoms, waxes, butters, ceramides, solid monoglycerides, diglycerides or triglycerides, and mixtures thereof.

The term “fatty acid” means a long-chain carboxylic acid comprising from 6 to 40 carbon atoms, preferably from 8 to 30 carbon atoms. The solid fatty acids according to the invention preferentially comprise from 10 to 30 carbon atoms and better still from 14 to 22 carbon atoms. These fatty acids are neither oxyalkylenated nor glycerolated.

The solid fatty acids that can be used in the present invention are notably chosen from myristic acid, cetylic acid, stearylic acid, palmitic acid, stearic acid, lauric acid, behenic acid, and mixtures thereof.

The term “fatty alcohol” means a long-chain aliphatic alcohol comprising from 6 to 40 carbon atoms, preferably from 8 to 30 carbon atoms, and comprising at least one hydroxyl group OH. These fatty alcohols are neither oxyalkylenated nor glycerolated.

The solid fatty alcohols may be saturated or unsaturated, and linear or branched, and include from 8 to 40 carbon atoms, preferably from 10 to 30 carbon atoms, better still from 12 to 30 carbon atoms. Preferably, the solid fatty alcohols have the structure R—OH with R denoting a linear alkyl group, optionally substituted with one or more hydroxyl groups, comprising from 8 to 40, preferentially from 10 to 30 carbon atoms, better still from 12 to 30, or even from 12 to 24 atoms and even better still from 14 to 22 carbon atoms.

The solid fatty alcohols that may be used are preferably chosen from saturated, and linear or branched, preferably linear and saturated, (mono) alcohols including from 8 to 40 carbon atoms, better still from 10 to 30, or even from 12 to 24 atoms and better still from 14 to 22 carbon atoms.

The solid fatty alcohols that may be used may be chosen, alone or as a mixture, from:
myristyl alcohol (or 1-tetradecanol);
cetyl alcohol (or 1-hexadecanol);
stearyl alcohol (or 1-octadecanol);
arachidyl alcohol (or 1-eicosanol);
behenyl alcohol (or 1-docosanol);
lignoceryl alcohol (or 1-tetracosanol);
ceryl alcohol (or 1-hexacosanol);
montanyl alcohol (or 1-octacosanol);
myricyl alcohol (or 1-triacontanol).

Preferentially, the solid fatty alcohol is chosen from cetyl alcohol, stearyl alcohol, behenyl alcohol, myristyl alcohol, arachidyl alcohol, and mixtures thereof, such as cetylstearyl or cetearyl alcohol. Particularly preferably, the solid fatty alcohol is chosen from cetyl alcohol, stearyl alcohol or mixtures thereof such as cetylstearyl alcohol, better still the solid fatty alcohol is cetylstearyl alcohol.

The solid esters of a fatty acid and/or of a fatty alcohol that may be used are preferably chosen from esters derived from a C₉-C₂₆ carboxylic fatty acid and/or from a C₉-C₂₆ fatty alcohol, different from esters of a linear or branched, saturated carboxylic acid including at least 10 carbon atoms and of a linear or branched, saturated monoalcohol, including at least 10 carbon atoms.

Esters of C₃-C₂₂ mono-, di- or tricarboxylic acids and of C₁-C₂₂ alcohols and esters of mono-, di- or tricarboxylic acids and of C₂-C₂₆ di-, tri-, tetra- or pentahydroxy alcohols may also be used.

Mention may notably be made of cetyl lactate, stearyl octanoate, octyl octanoate, cetyl octanoate, decyl oleate, hexyl stearate, octyl stearate, octyl pelargonate, diethyl sebacate, diisopropyl sebacate, diisopropyl adipate, di-n-propyl adipate, dioctyl adipate, dioctyl maleate, octyl palmitate, and mixtures thereof.

For the purposes of the present invention, a wax is a lipophilic compound, which is solid at 25° C. and atmospheric pressure, with a reversible solid/liquid change of state, having a melting point greater than about 40° C. and which may range up to 200° C., and having, in the solid state, an anisotropic crystal organization. In general, the size of the wax crystals is such that the crystals diffract and/or scatter light, giving the composition that comprises them a more or less opaque cloudy appearance. By bringing the wax to its melting point, it is possible to make it miscible with oils and to form a microscopically homogeneous mixture, but on returning the temperature of the mixture to ambient temperature, recrystallization of the wax, which is microscopically and macroscopically detectable (opalescence), is obtained.

In particular, the waxes that are suitable for use in the invention may be chosen from waxes of animal, plant or mineral origin, non-silicone synthetic waxes, and mixtures thereof.

Mention may notably be made of hydrocarbon-based waxes, for instance beeswax or modified beeswaxes (cera bellina), lanolin wax and lanolin derivatives, spermaceti; cork fibre or sugarcane waxes, olive tree wax, rice bran wax, carnauba wax, candelilla wax, ouricury wax, esparto grass wax, berry wax, shellac wax, Japan wax and sumac wax, absolute waxes of flowers; montan wax, orange wax, lemon wax, microcrystalline waxes, paraffins, petroleum jelly, lignite and ozokerite; polyethylene waxes, the waxes obtained by Fischer-Tropsch synthesis and waxy copolymers, and also esters thereof.

Mention may also be made of C₂ to C₆₀ microcrystalline waxes, such as Microwax HW.

Mention may also be made of the PM 500 polyethylene wax sold under the reference Permalen 50-L polyethylene.

Mention may also be made of the waxes obtained by catalytic hydrogenation of animal or plant oils containing linear or branched C₈ to C₃₂ fatty chains. Among these waxes mention may notably be made of isomerized jojoba oil such as the trans-isomerized partially hydrogenated jojoba oil, notably the product manufactured or sold by the company Desert Whale under the commercial reference Iso-Jojoba-50®, hydrogenated sunflower oil, hydrogenated castor oil, hydrogenated coconut kernel oil, hydrogenated lanolin oil and bis (1,1,1-trimethylolpropane) tetrastearate, notably the product sold under the name Hest 2T-4S® by the company Heterene.

The waxes obtained by hydrogenation of castor oil esterified with cetyl alcohol, such as those sold under the names Phytowax Castor 16L64® and 22L73® by the company Sophim, may also be used.

A wax that may also be used is a C₂₀ to C₄₀ alkyl (hydroxystearyloxy) stearate (the alkyl group comprising from 20 to 40 carbon atoms), alone or as a mixture. Such a wax is notably sold under the names Kester Wax K 82 P®, Hydroxypolyester K 82 P® and Kester Wax K 80 P® by the company Koster Keunen.

It is also possible to use microwaxes in the compositions of the invention; mention may notably be made of carnauba microwaxes, such as the product sold under the name MicroCare 350® by the company Micro Powders, synthetic-wax microwaxes, such as the product sold under the name MicroEase 114S® by the company Micro Powders, microwaxes constituted of a mixture of carnauba wax and polyethylene wax, such as the products sold under the names Micro Care 300® and 310® by the company Micro Powders, microwaxes constituted of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene microwaxes, such as the products sold under the names Microslip 519® and 519 L® by the company Micro Powders.

The waxes are preferably chosen from mineral waxes, for instance paraffin, petroleum jelly, lignite or ozokerite wax; plant waxes, for instance cocoa butter, shea butter or cork fibre or sugar cane waxes, olive tree wax, rice bran wax, hydrogenated jojoba wax, ouricury wax, carnauba wax, candelilla wax, esparto grass wax, or absolute waxes of flowers, such as the essential wax of blackcurrant blossom sold by the company Bertin (France); waxes of animal origin, for instance beeswaxes or modified beeswaxes (cera bellina), spermaceti, lanolin wax and lanolin derivatives; microcrystalline waxes; and mixtures thereof.

Butters may also be used.

For the purposes of the present invention, the term “butter” (also referred to as “pasty fatty substance”) is intended to mean a lipophilic fatty compound with a reversible solid/liquid change of state, comprising at a temperature of 25° C. and at atmospheric pressure (760 mmHg), a liquid fraction and a solid fraction. Preferably, the butter(s) according to the invention have a melting start temperature above 25° C. and a melting end temperature below 60° C.

Preferably, the particular butter(s) are of plant origin, such as those described in Ullmann's Encyclopedia of Industrial Chemistry (“Fats and Fatty Oils”. A. Thomas, published online: 15 Jun. 2000, DOI: 10.1002/14356007.a10_173, point 13.2.2.2. Shea Butter, Borneo Tallow, and Related Fats (Vegetable Butters)).

Mention may be made more particularly of shea butter, Nilotica shea butter (Butyrospermum parkii), galam butter (Butyrospermum parkii), Borneo butter or fat or tengkawang tallow (Shorea stenoptera), shorea butter, illipé butter, madhuca butter or Bassia madhuca longifolia butter, mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya butter (Kpangnan) (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter and sunflower butter.

An example of a preferred butter is shea butter.

In a known manner, shea butter is extracted from the fruit (also called “kernels” or “nuts”) of the Butyrospermum parkii tree. Each fruit contains between 45% and 55% fatty substance, which is generally extracted and refined.

Shea butter contains fatty acid triglycerides.

The fatty acid distribution of shea butter is variable, notably depending on the geographical origin of the nuts, the total percentage of stearic and oleic acids being very greatly predominant and generally greater than 80% by weight relative to the total weight of fatty acids.

The ceramides, or ceramide analogues such as glycoceramides, that may be used in the compositions according to the invention, are known; mention may be made in particular of ceramides of classes I, II, III and V according to the Dawning classification.

The ceramides or analogues thereof that may be used preferably correspond to the following formula:

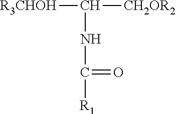

in which:
R₁ denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R₂ denotes a hydrogen atom, a (glycosyl) n group, a (galactosyl) m group or a sulfogalactosyl group, in which n is an integer ranging from 1 to 4 and m is an integer ranging from 1 to 8;
R₃ denotes a C₁₅-C₂₆ hydrocarbon-based group, which is saturated or unsaturated in the alpha position, it being possible for this group to be substituted with one or more C₁-C₁₄ alkyl groups;
it being understood that, in the case of natural ceramides or glycoceramides, R₃ may also denote a C₁₅-C₂₆ alpha-hydroxyalkyl group, the hydroxyl group optionally being esterified with a C₁₆-C₃₀ alpha-hydroxy acid.

Preferentially, use is made of ceramides for which R₁ denotes a saturated or unsaturated alkyl group derived from C₁₄-C₃₀ fatty acids; R₂ denotes a galactosyl or sulfogalactosyl group; and R₃ denotes a —CH═CH—(CH₂)₁₂—CH₃ group.

The ceramides that are more particularly preferred are the compounds for which R₁ denotes a saturated or unsaturated alkyl derived from C₁₆-C₂₂ fatty acids; R₂ denotes a hydrogen atom and R₃ denotes a saturated or unsaturated linear Cis group.

Use may also be made of the compounds for which R₁ denotes a saturated or unsaturated alkyl radical derived from C₁₂-C₂₂ fatty acids; R₂ denotes a galactosyl or sulfogalactosyl radical and R₃ denotes a saturated or unsaturated C₁₂-C₂₂ hydrocarbon-based radical and preferably a —CH═CH—(CH₂)₁₂—CH₃ group.

As compounds that are particularly preferred, mention may also be made of 2-N-linoleoylaminooctadecane-1,3-diol; 2-N-oleoylaminooctadecane-1,3-diol; 2-N-palmitoylaminooctadecane-1,3-diol; 2-N-stearoylaminooctadecane-1,3-diol; 2-N-behenoylaminooctadecane-1,3-diol; 2-N-[2-hydroxypalmitoyl]aminooctadecane-1,3-diol; 2-N-stearoylaminooctadecane-1,3,4-triol and in particular N-stearoylphytosphingosine, 2-N-palmitoylaminohexadecane-1,3-diol, N-linoleoyldihydrosphingosine, N-oleoyldihydrosphingosine, N-palmitoyldihydrosphingosine, N-stearoyldihydrosphingosine, and N-behenoyldihydrosphingosine, N-docosanoyl-N-methyl-D-glucamine, cetylic acid N-(2-hydroxyethyl)-N-(3-cetyloxy-2-hydroxypropyl) amide and bis(N-hydroxyethyl-N-cetyl) malonamide; and mixtures thereof. N-Oleoyldihydrosphingosine will preferably be used.

Preferably, the additional fatty substance(s) with a melting point above 25° C. (ii) are chosen from solid fatty alcohols, waxes, butters and mixtures thereof, more preferentially from solid fatty alcohols, butters and mixtures thereof, more preferentially still from cetyl alcohol, stearyl alcohol, cetearyl alcohol, shea butter and mixtures thereof.

Advantageously, in the composition according to the invention, the total content of the fatty substance(s) with a melting point above 25° C. (ii), different from fatty amines (i), ranges from 6% to 20% by weight, preferably from 7% to 15% by weight, preferentially from 8% to 12% by weight, relative to the total weight of the composition.

Advantageously, in the composition according to the invention, the total content of the fatty substance(s) with a melting point above 25° C. (ii), different from fatty amines (i), chosen from esters of a linear or branched, saturated carboxylic acid including at least 10 carbon atoms and of a linear or branched, saturated monoalcohol, including at least 10 carbon atoms and of additional solid fatty substances (ii), ranges from 6% to 20% by weight, preferably from 7% to 15% by weight, preferentially from 8% to 12% by weight, relative to the total weight of the composition.

Advantageously, in the composition according to the invention, the total content of the fatty substance(s) with a melting point above 25° C. (ii), different from fatty amines (i), i.e of esters of a linear or branched, saturated carboxylic acid including at least 10 carbon atoms and of a linear or branched, saturated monoalcohol, including at least 10 carbon atoms and of additional solid fatty substances (ii) chosen from solid fatty alcohols, ranges from 6% to 20% by weight, preferably from 7% to 15% by weight, preferentially from 8% to 12% by weight, relative to the total weight of the composition.

Advantageously, the weight ratio between the total content of the fatty amine(s) containing at least one C₈-C₃₀ hydrocarbon-based chain (i) and the total content of the fatty substance(s) with a melting point above 25° C. (ii), different from fatty amines (i), is between 0.1 and 1.0, preferably between 0.2 and 0.7.

In a preferred embodiment, the weight ratio between the total content of the fatty amine(s) containing at least one C₈-C₃₀ hydrocarbon-based chain (i) and the total content of the solid fatty alcohols, solid esters of fatty acids and/or of fatty alcohols, butters and mixtures thereof, is between 0.1 and 1.0, preferably between 0.2 and 0.7.

In another preferred embodiment, the weight ratio between the total content of the fatty amine(s) containing at least one C₈-C₃₀ hydrocarbon-based chain (i) and the total content of esters of a linear or branched, saturated carboxylic acid including at least 10 carbon atoms and of a linear or branched, saturated monoalcohol, including at least 10 carbon atoms and of additional solid fatty substances (ii), is between 0.1 and 1.0, preferably between 0.2 and 0.7.

In another preferred embodiment, the weight ratio between the total content of the (C₁₀-C₃₀) alkylamido (C₁-C₈) alkyl (di)(C₁-C₆) alkylamine(s) and the total content of the fatty substance(s) with a melting point above 25° C. (ii), different from fatty amines (i), is between 0.1 and 1.0, preferably between 0.2 and 0.7.

In yet another preferred embodiment, the weight ratio between the total content of the (C₁₀-C₃₀) alkylamido (C₁-C₈) alkyl (di)(C₁-C₆) alkylamine(s) and the total content of the solid fatty alcohols, solid esters of fatty acids and/or of fatty alcohols, butters and mixtures thereof, is between 0.1 and 1.0, preferably between 0.2 and 0.7.

In yet another preferred embodiment, the weight ratio between the total content of the (C₁₀-C₃₀) alkylamido (C₁-C₈) alkyl (di)(C₁-C₆) alkylamine(s) and the total content of esters of a linear or branched, saturated carboxylic acid including at least 10 carbon atoms and of a linear or branched, saturated monoalcohol, including at least 10 carbon atoms and of additional solid fatty substances (ii), is between 0.1 and 1.0, preferably between 0.2 and 0.7.

Liquid Fatty Substances (iii)

The cosmetic composition according to the present invention comprises at least one fatty substance (iii), different from fatty amines (i), with a melting point of less than or equal to 25° C., preferably less than or equal to 20° C., at atmospheric pressure (1.013×10⁵ Pa), preferably including fatty esters. In other words, these fatty substances are liquid at atmospheric pressure, and are not in the solid state.

In the present patent application, this or these fatty substances are also referred to as “liquid fatty substance(s)” or “oil(s)”.

Advantageously, the liquid fatty substances that can be used in the present invention are not (poly) oxyalkylenated.

Preferably, the liquid fatty substances that can be used in the present invention are non-silicone fatty substances.

The term “non-silicone fatty substance” refers to a fatty substance not containing any Si—O bonds and the term “silicone fatty substance” refers to a fatty substance containing at least one Si—O bond.

More particularly, the fatty substance(s) with a melting point below or equal to 25° C. (iii) according to the invention are chosen from C₆ to C₁₆ liquid hydrocarbons, liquid hydrocarbons comprising more than 16 carbon atoms, non-silicone oils of animal origin, oils of triglyceride type of plant or synthetic origin, fluoro oils, liquid fatty alcohols, liquid esters of fatty acid and/or fatty alcohol different from triglycerides, and mixtures thereof.

It is recalled that the fatty alcohols, esters and acids more particularly contain at least one saturated or unsaturated, linear or branched hydrocarbon-based group, comprising from 6 to 40, better still from 8 to 30 carbon atoms, which is optionally substituted, in particular, with one or more hydroxyl groups (in particular 1 to 4). If they are unsaturated, these compounds may comprise one to three conjugated or non-conjugated carbon-carbon double bonds.

As regards the C₆ to C₁₆ liquid hydrocarbons, the latter may be linear, branched, or optionally cyclic, and are preferably chosen from alkanes. Examples that may be mentioned include hexane, cyclohexane, undecane, dodecane, isododecane, tridecane or isoparaffins, such as isohexadecane or isodecane, and mixtures thereof.

The liquid hydrocarbons comprising more than 16 carbon atoms may be linear or branched, and of mineral or synthetic origin, and are preferably chosen from liquid paraffins or liquid petroleum jelly, polydecenes, hydrogenated polyisobutene such as Parleam®, and mixtures thereof.

Among the hydrocarbon-based oils of animal origin, mention may be made of perhydrosqualene.

The triglyceride oils of plant or synthetic origin are preferably chosen from liquid fatty acid triglycerides including from 6 to 30 carbon atoms, for instance heptanoic or octanoic acid triglycerides, or alternatively, for example, sunflower oil, maize oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearinerie Dubois or those sold under the names Miglyol® 810, 812 and 818 by the company Dynamit Nobel, jojoba oil and shea butter oil, and mixtures thereof.

As regards the fluoro oils, they may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethylperfluoromorpholine sold under the name PF 5052® by the company 3M.

The liquid fatty alcohols that are suitable for use in the invention are more particularly chosen from linear or branched, saturated or unsaturated alcohols, preferably unsaturated or branched alcohols, comprising from 6 to 40 carbon atoms, preferably from 8 to 30 carbon atoms. Examples that may be mentioned include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol and linoleyl alcohol, and mixtures thereof.

As regards the liquid fatty acid and/or fatty alcohol esters, different from the triglycerides mentioned previously, mention may be made notably of esters of saturated or unsaturated, linear C₁ to C₂₆ or branched C₃ to C₂₆ aliphatic monoacids or polyacids and of saturated or unsaturated, linear C₁ to C₂₆ or branched C₃ to C₂₆ aliphatic monoalcohols or polyalcohols, the total carbon number of the esters being greater than or equal to 6 and more advantageously greater than or equal to 10.

Preferably, for the esters of monoalcohols, at least one from among the alcohol and the acid from which the esters of the invention are derived is branched.

Among the monoesters, mention may be made of dihydroabictyl behenate; octyldodecyl behenate; isocetyl behenate; isostearyl lactate; lauryl lactate; linoleyl lactate; oleyl lactate; isostearyl octanoate; isocetyl octanoate; octyl octanoate; decyl oleate; isocetyl isostearate; isocetyl laurate; isocetyl stearate; isodecyl octanoate; isodecyl oleate; isononyl isononanoate; isostearyl palmitate; methyl acetyl ricinoleate; octyl isononanoate; 2-ethylhexyl isononanoate; octyldodecyl erucate; oleyl erucate; ethyl palmitate, isopropyl palmitate, 2-ethylhexyl palmitate, 2-octyldecyl palmitate; alkyl myristates such as isopropyl myristate, 2-octyldodecyl myristate; isobutyl stearate; 2-hexyldecyl laurate, and mixtures thereof.

Preferably, among the monoesters of monoacids and of monoalcohols, use will be made of ethyl palmitate or isopropyl palmitate, alkyl myristates such as isopropyl myristate or ethyl myristate, isocetyl stearate, 2-ethylhexyl isononanoate, isodecyl neopentanoate and isostearyl neopentanoate, and mixtures thereof.

Still within the context of this variant, esters of C₄ to C₂₂ dicarboxylic or tricarboxylic acids and of C₁ to C₂₂ alcohols and esters of mono-, di- or tricarboxylic acids and of C₂ to C₂₆ di-, tri-, tetra- or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates, and mixtures thereof.

The composition may also comprise, as fatty ester, sugar esters and diesters of C₆ to C₃₀, preferably C₁₂ to C₂₂, fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be chosen notably from the group comprising the esters or mixtures of esters of sugars described previously and of linear or branched, saturated or unsaturated C₆ to C₃₀ and preferably C₁₂ to C₂₂ fatty acids. If they are unsaturated, these compounds may comprise one to three conjugated or non-conjugated carbon-carbon double bonds.

The esters according to this variant may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates, arachidonates or mixtures thereof notably such as the mixed oleo-palmitate, oleo-stearate and palmito-stearate esters.

More particularly, use is made of monoesters and diesters and notably sucrose, glucose or methylglucose mono- or di-oleates, -stearates, -behenates, -oleopalmitates, -linoleates, -linolenates and -oleostearates, and mixtures thereof.

An example that may be mentioned is the product sold under the name Glucate® DO by the company Amerchol, which is a methylglucose dioleate.

Preferably, use will be made of a liquid ester of a monoacid and of a monoalcohol.

Preferably, the fatty substance(s) with a melting point below or equal to 25° C. (iii), different from the fatty amines (i), are chosen from triglyceride oils of plant or synthetic origin, liquid fatty acid and/or fatty alcohol esters different from triglycerides, and mixtures thereof, and more preferentially from sunflower oil, soybean oil, olive oil, avocado oil, isopropyl myristate, and mixtures thereof.

Advantageously, in the composition according to the invention, the total content of the fatty substance(s) with a melting point below or equal to 25° C. (iii), different from the fatty amines (i), ranges from 0.1% to 10% by weight, preferably from 0.5% to 8% by weight, more preferentially from 1% to 5% by weight, relative to the total weight of the composition.

Preferably, the composition comprises:
one or more fatty substances with a melting point above 25° C. (ii) chosen from esters of a linear or branched, saturated carboxylic acid including at least 10 carbon atoms and of a linear or branched, saturated monoalcohol, including at least 10 carbon atoms,
one or more fatty substance(s) with a melting point below or equal to 25° C. (iii) chosen from triglyceride oils of plant origin.

More preferably, the composition comprises:
one or more fatty substances with a melting point above 25° C. (ii) chosen from esters of a linear or branched, saturated carboxylic acid including at least 10 carbon atoms and of a linear or branched, saturated monoalcohol, including at least 10 carbon atoms,
one or more fatty substances with a melting point above 25° C. (ii) chosen from solid fatty alcohols,
one or more fatty substance(s) with a melting point below or equal to 25° C. (iii) chosen from triglyceride oils of plant origin.

The composition according to the invention generally comprises water.

The water advantageously represents from 65% to 95% by weight, better still from 70% to 90% by weight, preferably from 75% to 85% by weight, relative to the total weight of the composition.

### Organic Solvents

The composition according to the invention may also comprise, furthermore, one or more water-miscible organic solvents.

Preferably, the organic solvent(s) are chosen from non-aromatic C₁-C₆ alcohols such as ethyl alcohol or isopropyl alcohol, aromatic alcohols such as benzyl alcohol and phenylethyl alcohol, or polyols such as glycerol or sorbitol.

When they are present in the composition according to the invention, the organic solvent(s) generally represent from 0.1% to 15% by weight and preferably from 0.5% to 10% by weight relative to the total weight of the composition.

### Emulsion

According to a preferred embodiment, the composition according to the invention is in the form of an oil-in-water emulsion.

When it is in the form of an emulsion, the composition according to the invention generally has very low polydispersity, i.e. the particles have a very homogeneous size. The particles present in the composition according to the invention are liquid oil (or oily phase) particles within the continuous aqueous phase.

### Additives

The cosmetic composition according to the present invention may also optionally comprise one or more additives, different from the compounds of the invention and among which mention may be made of surfactants different from the fatty amines, such as cationic, anionic, nonionic or amphoteric surfactants, and mixtures thereof, cationic, anionic, nonionic or amphoteric polymers, or mixtures thereof, antidandruff agents, antiseborrhoeic agents, vitamins and provitamins including panthenol, sunscreens, sequestrants, plasticizers, solubilizers, acidifying agents, mineral or organic thickeners, in particular polymeric thickeners, opacifiers, pearlescent agents, antioxidants, hydroxy acids, fragrances, preserving agents and fillers.

Needless to say, those skilled in the art will take care to select this or these optional additional compounds such that the advantageous properties intrinsically associated with the composition according to the invention are not, or are not substantially, adversely affected by the envisaged addition(s).

The above additives may generally be present in an amount, for each of them, of between 0 and 20% by weight relative to the total weight of the composition.

A subject of the invention is also a process for the cosmetic treatment of human keratin fibres such as the hair, comprising the application to said keratin fibres of a composition as defined above.

The composition according to the invention may be applied to dry or wet keratin materials that have optionally been washed with shampoo. Preferably, the composition according to the invention is applied to wet keratin fibres.

According to one embodiment, the keratin fibres are then rinsed with water, and optionally washed with a shampoo and then rinsed with water, before being dried or left to dry.

In this embodiment, the composition according to the invention is applied for a leave-on time that may range from 1 to 15 minutes and preferably from 2 to 10 minutes.

According to one preferred embodiment, the keratin fibres are not rinsed after application of the composition. They can then undergo heat and/or mechanical treatment for drying and/or shaping, for example straightening by means of a heating iron.

A subject of the invention is also the use of a composition as defined above, for conditioning keratin fibres, in particular human keratin fibres such as the hair.

The composition can be used on wet or dry hair, in rinse-off or leave-on mode, and preferably in leave-on mode (that is to say that the keratin fibres are not rinsed after application of the composition).

The examples which follow serve to illustrate the invention without, however, exhibiting a limiting nature.

### EXAMPLES

In the examples that follow, all the amounts are given, unless otherwise indicated, as mass percentages of active material (AM) relative to the total weight of the composition.

### Example 1

The compositions A and B according to the invention were prepared from the following ingredients.

TABLE 1

| | Composition | Composition |
|---|---|---|
| | A | B |
| Isopropyl myristate | 2 | 2 |
| Caprylyl glycol | 0.3 | 0.3 |
| Cetyl esters | 1.5 | 1.5 |
| Cetearyl alcohol | 9 | 6 |
| Shea butter | — | 1 |
| Stearamidopropyl | 4.74 | 2.37 |
| dimethylamine | | |
| Sunflower oil | 1 | 1 |
| Tartaric acid | 0.9 | 0.42 |
| Preserving agents | qs | qs |
| Water | qs 100 | qs 100 |

Compositions which can be used as a hair care composition (conditioner) have been obtained.

1 g of composition A or B was applied to a 2.7 g lock of wet hair (damaged hair) which had previously been shampooed.

The product was left on for 5 minutes then the lock was rinsed with hot water (38° C.) for 15 seconds.

The lock was then dried using a hairdryer.

Compositions A and B according to the invention made it possible to obtain very good cosmetic properties, which were at least equivalent to those obtained with a silicone non-natural haircare composition (conditioner).

In particular, the compositions according to the invention were easy to apply and to spread on the keratin fibres.

These compositions also had a creamy texture.

Compositions A and B made it possible to improve the disentangling of the head of hair, and to give the keratin fibres a smooth and soft feel. They also gave the hair manageability.

Compositions A and B according to the invention were particularly suitable for conditioning damaged hair.

### Example 2

Composition C according to the invention and comparative composition C′ were prepared from the following ingredients (g % AM):

TABLE 2

| | Composition C | Composition C′ |
|---|---|---|
| | (invention) | (comparative) |
| Stearamidopropyl dimethylamine | 2.4 | 2.4 |
| Cetearyl alcohol | 6.0 | 3.0 |
| Cetyl esters | 1.5 | 1.0 |
| Isopropyl myristate | 2.0 | 5.5 |
| Sunflower oil | 1.0 | 1.0 |
| Shea butter | 1.0 | 1.0 |
| Caprylyl glycol | 0.3 | 0.3 |
| Tartaric acid | 0.4 | 0.4 |
| Preserving agents | qs | qs |
| Water | qs 100 | qs 100 |

Compositions which can be used as a hair care composition (conditioner) were obtained.

0.4 g of composition per g of hair was applied, to wet locks of SA20 hair (damaged hair), previously washed with a shampoo and rinsed.

The product was left on for 3 minutes then the lock was rinsed with hot water (38° C.) for 15 seconds.

The two compositions were evaluated under blind conditions by 5 evaluators, who gave each one a score from 0 to 5 (in steps of 0.5), for the following criteria:
Suppleness on application: ability of the composition to make the fibres more supple,
Smooth feel after rinsing: ease of sliding the fingers along the lock.

The following results were obtained:

TABLE 3

| | | Evaluator | Evaluator | Evaluator | Evaluator | Evaluator | | Standard |
|---|---|---|---|---|---|---|---|---|
| Criterion | | 1 | 2 | 3 | 4 | 5 | Mean | deviation |
| Suppleness | C (invention) | 3 | 4 | 3.5 | 3 | 3.5 | 3.4 | 0.37 |
| | C′ (comparative) | 2.5 | 2 | 2 | 2.5 | 3 | 2.4 | 0.37 |
| Smooth | C (invention) | 3.5 | 4 | 4 | 4 | 4.5 | 4 | 0.32 |
| feel | C′ (comparative) | 2 | 0.5 | 2 | 2.5 | 3 | 2 | 0.84 |

It is observed that, in 100% of the cases, the composition of the invention C made it possible to obtain significantly higher results as regards the suppleness of the fibre and the smooth feel thereof, compared to the comparative composition C′.

### Example 3

Compositions A1 and A2 according to the invention and comparative compositions B1 and B2 were prepared from the following ingredients (g % AM):

TABLE 4

| | Composition A1 | Composition B1 |
|---|---|---|
| | (invention) | (comparative) |
| Cetearyl alcohol | 8 | 9 |
| Benzyl alcohol | 0.5 | 0.3 |
| Butyrospermum parkii (shea) | 1 | — |
| butter/Butyrospermum parkii | | |
| butter | | |
| Cetyl esters* (INCI name) | 1 | — |
| Stearamidopropyl dimethylamine | 4.74 | 4.74 |
| Tartaric acid | 0.9 | 0.9 |
| Isopropyl myristate | 2 | 2 |
| Helianthus annuus (Sunflower) | 1 | 1 |
| seed oil/Helianthus annuus seed | | |
| oil | | |
| Preserving agents/perfume | qs | qs |
| Water | qs 100 | qs 100 |

*mixture of esters of fatty acids having 14 to 18 carbon atoms and of cetyl alcohol

TABLE 5

| | Composition A2 | Composition B2 |
|---|---|---|
| | (invention) | (comparative) |
| Cetearyl alcohol | 9 | 9 |
| Benzyl alcohol | 0.3 | 0.3 |
| Cetyl esters* (INCI name) | 1 | — |
| Stearamidopropyl dimethylamine | 4.74 | 4.74 |
| Tartaric acid | 0.9 | 0.9 |
| Isopropyl myristate | 2 | 2 |
| Helianthus annuus (Sunflower) | 1 | 1 |
| seed oil/Helianthus annuus seed | | |
| oil | | |
| Preserving agents/perfume | qs | qs |
| Water | qs 100 | qs 100 |

*mixture of esters of fatty acids having 14 to 18 carbon atoms and of cetyl alcohol

### Protocol:

Each of the compositions A1, A2, B1 and B2 was applied in a standardized manner, on wet natural hair strands, previously washed, at a rate of 0.4 g of composition per gram of hair lock.

Each composition was left on for about 15 seconds, and then the hair locks were rinsed with water for 15 seconds. The hair strands were then wrung out by passing them between 2 fingers, evaluated wet then laid flat and allowed to air dry, before being evaluated again.

Composition A1 was compared to composition B1 and composition A2 was compared to composition B2.

### Performance on Wet Hair: Ease of Distribution, Rich Touch

The impact of the invention on the ease of distribution and the rich touch of the hair was evaluated under blind conditions by 2 experts, on wet hair.

Each expert elected the composition that presented the easiest and most homogeneous distribution.

The compositions A1 and A2 according to the invention are respectively easier to distribute on the surface of the hair strand than the comparative compositions B1 and B2

The experts then elected the composition that provided the most rich feel to the hair, by tactilely evaluating the coating and softening of the hair immediately after application.

The hair treated with compositions A1 and A2 according to the invention respectively had a more rich feel (more coated, more softened) than those treated with the comparative compositions B1 and B2.

### Performance on Dry Hair: Smooth and Homogeneous Touch

The impact of the invention on the smooth and homogeneous touch of the hair was evaluated by 2 experts under blind conditions on dry hair: each of the 2 experts elected the lock that had the smoothest and most homogeneous touch.

In order to evaluate the smoothness and homogeneity of the touch, the expert took a lock of hair between the thumb and the index finger and slid their fingers along the lock from the roots to the ends;

They evaluated if the hair was soft, if there were no roughness, if it did not catch on the fingers, if the touch was homogeneous from the roots to the ends.

The compositions A1 and A2 according to the invention exhibited improved the performance in terms of smooth touch and homogeneity of dry hair compared to the comparative compositions B1 and B2.

1. Cosmetic composition comprising:
(i) one or more fatty amines containing at least one C₈-C₃₀ hydrocarbon-based chain,
(ii) at least 6% by weight, relative to the total weight of the composition, of one or more fatty substances with a melting point above 25° C., different from the fatty amines (i), and
(iii) one or more fatty substances with a melting point below or equal to 25° C., different from the fatty amines (i),
the composition comprising one or more fatty substances with a melting point above 25° C. (ii) chosen from esters of a linear or branched, saturated carboxylic acid including at least 10 carbon atoms and of a linear or branched, saturated monoalcohol, including at least 10 carbon atoms.

2. Composition according to claim 1, characterized in that the fatty amine(s) containing at least one C₈-C₃₀ hydrocarbon-based chain (i) are chosen from (C₁₀-C₃₀) alkylamido (C₁-C₈) alkyl (di)(C₁-C₆) alkylamines, salts thereof and solvates thereof; preferably from (C₁₄-C₂₆) alkylamido (C₁-C₆) alkyl (di)(C₁-C₄) alkylamines, salts thereof and solvates thereof; more preferentially from (C₁₆-C₂₄) alkylamido (C₂-C₄) alkyl (di)(C₁-C₂) alkylamines, salts thereof and solvates thereof; more preferentially still from (C₁₈-C₂₂) alkylamido (C₂-C₄) alkyl (di)(C₁-C₂) alkylamine, salts thereof and solvates thereof; better still from brassicamidopropyl dimethylamine, stearamidopropyl dimethylamine, behenamidopropyl dimethylamine, and mixtures thereof.

3. Composition according to claim 1, characterized in that the total content of the fatty amine(s) containing at least one C₈-C₃₀ hydrocarbon-based chain (i) is greater than or equal to 2% by weight, preferably ranges from 2% to 10% by weight, more preferentially ranges from 2% to 6% by weight, relative to the total weight of the composition.

4. Composition according to claim 1, characterized in that the esters of a linear or branched, saturated carboxylic acid including at least 10 carbon atoms and of a linear or branched, saturated monoalcohol, including at least 10 carbon atoms are chosen from esters of a linear or branched, saturated carboxylic acid including from 10 to 30 carbon atoms and preferably from 12 to 24 carbon atoms, and of a linear or branched, saturated monoalcohol, including from 10 to 30 carbon atoms and preferably from 12 to 24 carbon atoms.

5. Composition according to claim 1, characterized in that the esters of a linear or branched, saturated carboxylic acid including at least 10 carbon atoms and of a linear or branched, saturated monoalcohol, including at least 10 carbon atoms are chosen from myristyl stearate, cetyl stearate, stearyl stearate, cetyl myristate, myristyl myristate, stearyl myristate, myristyl palmitate, cetyl palmitate, stearyl palmitate, and mixtures thereof.

6. Composition according to claim 1, characterized in that it also comprises one or more additional fatty substance(s) with a melting point above 25° C. (ii) chosen from solid fatty alcohols, waxes, butters and mixtures thereof, preferably from solid fatty alcohols, butters and mixtures thereof, more preferentially from cetyl alcohol, stearyl alcohol, cetearyl alcohol, shea butter and mixtures thereof.

7. Composition according to claim 1, characterized in that the total content of the fatty substance(s) with a melting point above 25° C. (ii) ranges from 6% to 20% by weight, preferably from 7% to 15% by weight, more preferentially from 8% to 12% by weight, relative to the total weight of the composition.

8. Composition according to claim 1, characterized in that the weight ratio between the total content of the fatty amine(s) containing at least one C₈-C₃₀ hydrocarbon-based chain (i) and the total content of the fatty substance(s) with a melting point above 25° C. (ii) is between 0.1 and 1.0, preferably between 0.2 and 0.7.

9. Composition according to claim 1, characterized in that the fatty substance(s) with a melting point below or equal to 25° C. (iii) are chosen from triglyceride oils of plant or synthetic origin, liquid esters of fatty acid and/or fatty alcohol different from triglycerides, and mixtures thereof, and preferably from sunflower oil, soybean oil, olive oil, avocado oil, isopropyl myristate, and mixtures thereof.

10. Composition according to claim 1, characterized in that the total content of the fatty substance(s) with a melting point below or equal to 25° C. (iii) ranges from 0.1% to 10% by weight, preferably from 0.5% to 8% by weight, more preferentially from 1% to 5% by weight, relative to the total weight of the composition.

11. Process for the cosmetic treatment of human keratin fibres such as the hair, comprising the application to said keratin fibres of a composition as defined in claim 1.

12. Use of a composition according to claim 1, for conditioning keratin fibres, in particular human keratin fibres such as the hair.
